(19) United States
(12) Patent Application Publication
(10) Pub. No.: US 2008/0207751 A1
(43) Pub. Date: Aug. 28, 2008

(54) Sulindac Derivatives for Treatment of Cancer

(75) Inventors: Anna Sparatore, Milan (IT); Piero Del Soldato, Monza (IT)

Correspondence Address: SCHNECK & SCHNECK, P.O. BOX 2-E, SAN JOSE, CA, 95109-0005, US

(73) Assignee: Sulfidris s.r.l., Milan (IT)

(21) Appl. No.: 11/917,321

(22) PCT Filed: Jun. 12, 2006
(86) PCT No.: PCT/IB06/01679

§ 371 Date: Jan. 30, 2008

(30) Foreign Application Priority Data
Jun. 14, 2005 (IT) MI2005A001107

(51) Int. Cl. A61K 31/216; C07C 69/73; A61P 35/00

(52) U.S. Cl. 514/532; 560/10

(57) ABSTRACT

The present invention relates to novel non steroidal anti-inflammatory compounds (NSAIDs) derivatives of sulindac, for the treatment/prevention, alone or in combination, of cancer.

### FIELD OF THE INVENTION

The present invention is related to new compounds as Sulindac derivatives for the treatment of cancer.

### BACKGROUND

The anti-inflammatory, analgesic and antipyretic drugs are an heterogeneous group of compounds, often chemically unrelated, which nevertheless share certain therapeutic actions and side effects. They are frequently called non steroidal anti-inflammatory drugs or NSAIDs.

Although NSAIDs had been known to inhibit a wide variety of reactions in vitro, the first convincing relationship was established by Vane et al. in 1971 when they demonstrated that low doses of aspirin and indomethacin inhibited the enzymatic production of prostaglandins. The first enzyme in the prostaglandin synthetic pathway is prostaglandin endoperoxide synthase, or fatty acid cyclooxygenase. It is now appreciated that there are two forms of cyclooxygenase termed cyclooxygenase-1 (COX-1) and cyclooxygenase-2 (COX-2).

More specifically at the beginning it was generally recognized that the beneficial actions of NSAIDs can be associated with inhibition of COX-2 whereas their harmful side effects are associated with inhibition of COX-1.

It is known from epidemiological studies that the taking of conventional NSAIDS (non-steroidal anti-inflammatory drugs, which are COX-1 and COX-2 inhibitors), the most common of which is aspirin, and the incidence of colon cancer have an inverse correlation. In addition, there have been many reports that NSAIDS, such as aspirin and sulindac, have shown inhibitory activity against tumor metastasis and carcinogenesis in preclinical studies.

Some NSAIDS, and among them sulindac, have been used in clinical studies for the prevention of colon carcinogenesis.

Sulindac is the name of a compound that can be present as sulfoxide ((Z)-5-Fluoro-2-methyl-1-[[4-(methylsulfinyl)phenyl]methylene]-1H-indene-3-acetic acid), sulfide ((Z)-5-Fluoro-2-methyl-1-[[4-(methylsulfide)phenyl]methylene]-1H-indene-3-acetic acid) and sulfone ((Z)-5-Fluoro-2-methyl-1-[[4-(methylsulfone)phenyl]methylene]-1H-indene-3-acetic acid). Sulindac sulfoxide is used as a precursor of sulindac sulfide that is formed in vivo and that is known to have a strong anti-inflammatory activity as it is a strong inhibitor of COX-1 and COX-2.

However, since conventional NSAIDS are not selective for COX-1 or COX-2, the occurrence of adverse effects, mainly gastrointestinal bleeding, gastrointestinal perforation, renal and liver toxicity, is unavoidable. NSAIDs are responsible for at least 100,000 hospitalizations and 10,000 to 20,000 deaths annually and are responsible for more serious adverse drug reactions reported to the FDA than any other class of drugs. These adverse effects are generally dose-dependent, with higher doses more likely to cause toxicity.

Selective COX-2 have addressed the problem of the gastrointestinal tolerability and greatly improved the gastric safety.

Among the known selective COX-2 inhibitors, it is known that celecoxib (Reddy, R. S. et al. “Evaluation of Cyclooxygenase-2 Inhibitor for Potential Chemopreventive Properties in colon Carcinogenesis”, Cancer Res., 56, 4566-4569 (1996)] inhibit the occurrence of experimental colonic polyposis. On the other hand the use of selective COX-2 inhibitors resulted in an increase of cardiovascular events after long term treatment.

It is known also that sulindac solfone is devoid of any activity as COX(s) inhibitor but an antitumoral activity, COX independent, in colon cancer has been recently reported. (Cancer Research 59, 3387-3391, 1999; Journal Biological Chemistry 278, 47762-47775, 2003).

The tumoral process seems to be characterized by the formation of a relevant inflammatory status involving many mediators, among whom, many cytokines, oxygen and nitrogen radical species (Ohshima H. et al. “Chemical basis of inflammation-induced carcinogenesis”. Arch. Biochem. Biophys. 417, 2003, 3-11).

Prostanoids that are inhibited by selective and non selective NSAIDs are only one of the many inflammatory mediators that are involved. It is therefore very important to broaden the number of the inflammatory mediators that are involved. Their formation is linked to the activation of the nuclear factors NFT).

In particular a fundamental role for the interaction between activatory and inhibitory subunits is played by the cysteinic residue.

Another important observation is that the metabolic activation of carcinogen substances is greatly reduced by the antioxidants enzymes of the phase 2 of the metabolic process (those acting on glutathion and thiol donors). It has been found that due to glutathione induction, or other antioxidants enzymes, it is possible to substantially reduce the gastrointestinal and hepatic toxicity of NSAIDs.

### SUMMARY

We have now found that certain derivatives of sulindac are effective for the treatment or prevention of tumour-related disorders, alone or in combination. Such compounds are efficacious and well tolerated.

### DETAILED DESCRIPTION

It is an object of the present invention sulindac derivatives having formula:

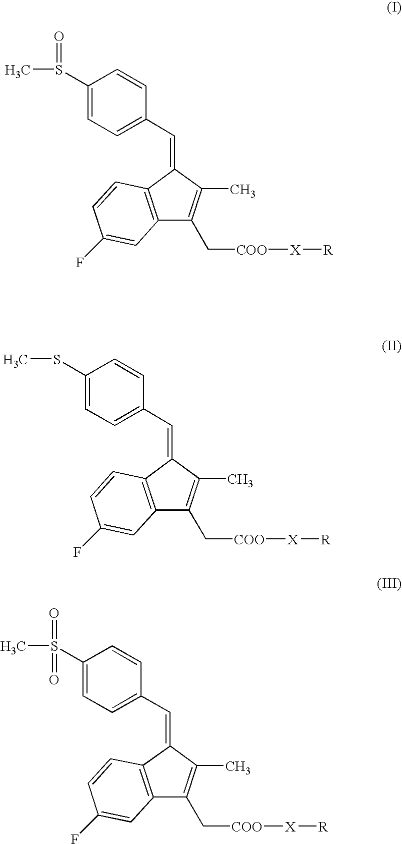

wherein X is absent or, if present, is selected among ethyl-, propyl-, butyl diols, dialkylamine, hydroxyalkylamine or linking groups such as esters, amides, imides, sulfonamides, azo groups, carbamates, carbonates, anhydrides, acetals, thioacetals and R is selected from the group comprising 5-(p-hydroxyphenyl)-3H-1,2-dithiol-3-thione, 1,3-dithiol-2-thione-5-carboxylic acid, 3-thioxo-3H-1,2-dithiole-5-carboxylic acid, 3-thioxo-3H-1,2-dithiole-4-carboxylic acid, hydroxyalkylmethanthiosulfonates, N-acetyl-penicillamine, S-allyl-cysteine, bucillamine, carbocysteine, cysteamine, cystathionine, homocysteine, mecysteine, methionine, pantetheine, penicillamine, penicillamine disulfide, thioacetic acid, thiodiglycolic acid, thioglycolic acid, thiolactic acid, 2-thiolhistidine, thiomalic acid, thiosalicylic acid, tiopronin.

As a further preferred embodiment of the sulindac derivatives according to the present invention, said sulindac derivatives are selected from the group comprising (Z)-5-Fluoro-2-methyl-1-[[4-(methylsulfinyl)phenyl]methylene]-1H-indene-3-acetic acid 4-(thioxo-5H-[1,2]dithiol-3-yl)-phenyl ester, (Z)-5-Fluoro-2-methyl-1-[[4-(methylsulfide)phenyl]methylene]-1H-indene-3-acetic acid 4-(thioxo-5H-[1,2]dithiol-3-yl)-phenyl ester, (Z)-5-Fluoro-2-methyl-1-[[4-(methylsulfone)phenyl]methylene]-1H-indene-3-acetic acid 4-(thioxo-5H-[1,2]dithiol-3-yl)-phenyl ester, (Z)-5-Fluoro-2-methyl-1-[[4-(methylsulfinyl)phenyl]methylene]-1H-indene-3-acetic acid 2-methanesulfonylsulfanylethyl ester, (Z)-5-Fluoro-2-methyl-1-[[4-(methylsulfone)phenyl]methylene]-1H-indene-3-acetic acid 2-methanesulfonylsulfanylethyl ester, (Z)-5-Fluoro-2-methyl-1-[[4-(methylsulfide)phenyl]methylene]-1H-indene-3-acetic acid 2-methanesulfonylsulfanylethyl ester.

It is a further object of the present invention the use of the above said sulindac derivatives (according to the present invention, as for general formulas (I), (II), (III) and selected derivatives as described above), as a medicament.

It is a further object of the present invention the use of the above said and described sulindac derivatives, as for general formulas (I), (II), (III) and selected derivatives as described above, for the manufacture of a medicament for the treatment and/or prevention of cancer, more preferably for the manufacture of a medicament for the treatment and/or prevention of colon cancer.

This invention relates to the field of cancer prevention and treatment with new derivatives of sulindac acting with multiple mechanisms of action. The new sulindac derivatives combine the mechanism of COX inhibition of nonsteroidal drugs with additional antitumoral mechanisms therefore increasing the activity and reducing toxicity. The derivatives compounds described in the present invention contain organic sulfur capable to capture electrophilic reactive species that act on the metabolic activation (transcription, linkage to the cellular DNA etc.)

Sulindac's (parent compound) activity (with exclusion of sulindac sulfone) is mainly dependent on the COX inhibition and this is the main mechanism for the anti-inflammatory/antitumoral activity and for its toxicity. The regulating part (SS) of the transcription factors with its differentiated effects of inhibition on NFkB and stimulation on nfr2 increases both the antinflammatory and antitumoral activity as well as the tolerability mainly at the gastrointestinal level. The regulator effects on transcription factors seem to be very relevant also for overcoming the resistance (Das K C et al. JBC 1997, 272, 14914; Fahy B N et al. J. Am. Coll. Surg. 2004, 198, 591) to the traditional antitumoral therapy and support the use of the new compounds of the present invention in anticancer co-therapy.

The parent compounds (i.e. the compounds in which the modification with NTF moieties can be applied) are sulindac sulfoxide (Ia), sulfide (IIa) or sulfone (IIIa), when —X—R═H, are reported below.

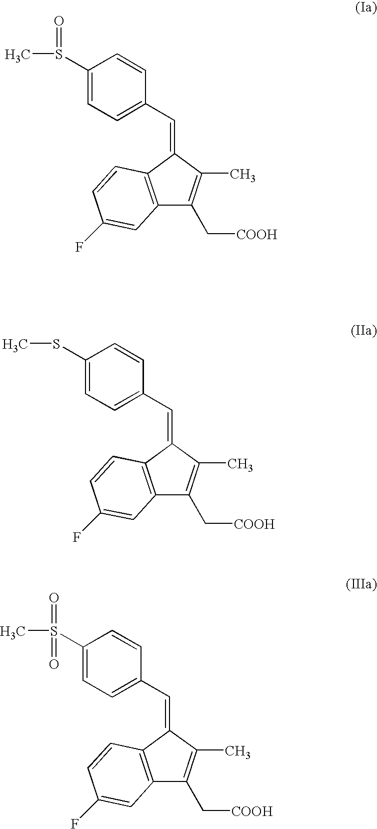

In the present invention the parent compound is considered in its original form or in a proper modification to allow the chemical manipulation with organic sulfur containing moieties, active on nuclear transcription factors, that can be attached directly or indirectly via a bi-functional linker group (X).

Substances (—R) containing organic sulfur active on nuclear transcription factors that can be linked to sulindac are 5-(p-hydroxyphenyl)-3H-1,2-dithiol-3-thione, 1,3-dithiol-2-thione-5-carboxylic acid, 3-thioxo-3H-1,2-dithiole-5-carboxylic acid, 3-thioxo-3H-1,2-dithiole-4-carboxylic acid. Also thiosulfonates are included in the organic sulfur containing moieties that can be linked to sulindac.

Other substances (—R) containing organic sulfur and that can be linked to sulindac are N-acetyl-penicillamine, S-allyl-cysteine, bucillamine, carbocysteine, cysteamine, cystathionine, homocysteine, mecysteine, methionine, pantetheine, penicillamine, penicillamine disulfide, thioacetic acid, thiodiglycolic acid, thioglycolic acid, thiolactic acid, 2-thiolhistidine, thiomalic acid, thiosalicylic acid, tiopronin.

The substances containing organic sulfur (—R) can be linked via different linking groups such as esters, amides, imides, sulfonamides, azo groups, carbamates, carbonates, anhydrides, acetals, thioacetals, etc.

Bi-functional linkers (X) known to the expert in the field (such as ethyl, propyl, or butyl diols; di-amines; hydroxyalkylamines, etc.) can be optionally present when they are necessary to link the drug to the NF-regulating moieties. (X) can not be present; in such a case (R) is linked directly to the parent compound.

Also salts pharmaceutically acceptable, containing organic sulfur, that directly or indirectly are capable to interact with transcription nuclear factors are part of the present invention.

When the compounds include at least one asymmetric carbon atom, the products can be used in racemic mixture or in form of single enantiomer.

The compounds of the present invention can be administered in the form of any pharmaceutical formulation, the nature of which will depend upon the route of administration and the nature of the disease to be treated. These pharmaceutical compositions can be prepared by conventional methods, using compatible, pharmaceutically acceptable excipients or vehicles. Examples of such compositions include capsules, tablets, syrups, powders and granulates for the preparation of extemporaneous solutions, injectable preparations, rectal, nasal, ocular, vaginal etc. A preferred route of administration is the oral route. The compounds of the present invention can be administered at doses between 1 and 60 mg/kg and preferably between 3 and 30 mg/kg.

It is a further object of the present invention the synthesis of Sulindac derivatives, as for general formulas (I), (II), (III) and selected derivatives as described above, said synthesis comprising the reaction of the parent compound Sulindac sulfoxide ((Z)-5-Fluoro-2-methyl-1-[[4-(methylsulfinyl)phenyl]methylene]-1H-indene-3-acetic acid), Sulindac sulfide ((Z)-5-Fluoro-2-methyl-1-[[4-(methylsulfide)phenyl]methylene]-1H-indene-3-acetic acid) or Sulindac sulfone ((Z)-5-Fluoro-2-methyl-1-[[4-(methylsulfone)phenyl]methylene]-1H-indene-3-acetic acid, respectively, with a substance (R) containing organic sulphur, active on nuclear transcription factors, the latter or the parent compound eventually modified with a bi-functional linker group (X), said substance (R) and the bi-functional linker group (X) being as defined above.

It is a further object of the present invention the use of Sulindac derivatives according to the present invention, as for general formulas (I), (II), (III) and selected derivatives as described above, for the treatment and/or prevention of cancer, more preferably for the treatment and/or prevention of colon cancer as well as the method for the treatment and/or prevention of cancer, more preferably for the treatment and/or prevention of colon cancer, said method comprising the administration of the Sulindac derivatives according to the present invention, as for general formulas (I), (II), (III) and selected derivatives as described above.

The following non-limitative examples further describe and enable an ordinary skilled in the art to make and use the invention.

### EXAMPLE 1

### Synthesis of (Z)-5-Fluoro-2-methyl-1-[[4-(methylsulfinyl)phenyl]methylene]-1H-indene-3-acetic acid 4-(thioxo-5H-1-[1,2]dithiol-3-yl)-phenyl ester

To 280 mmol of sulphur, 40 mmol of anethole in 20 ml of dimethylacetamide were added. After heating at 145° C. for 6 hours, 2.5 g of anethole dithiolethione (ADT) were obtained. The product, washed with ether, was crystallized by ethyl acetate: melting point 110-111° C. Then 1.5 g of ADT were mixed with 7.5 g of pyridine HCl and the mixture was heated for 25 minutes at 215° C. After cooling, 1N HCl in excess was added and the precipitate was filtered, washed and crystallized from ethanol. The obtained compound, 5-(p-hydroxyphenyl)-3H-1,2-ditiol-3-thione, melted at 191-192° C.

A 1N solution of dicyclohexylcarbodiimide (DCC, 612 mg) in dichloromethane (2.7 ml) is added to 100 ml of a dichloromethane solution containing 5-(p-hydroxyphenyl)-3H-1,2-dithiol-3-thione (610.2 mg, 2.7 mmol) prepared as above described, sulindac (601 mg, 2.7 mmol), and a catalytic amount (15 mg) of 4-dimethylaminopyridine (DMAP).

The mixture is stirred at room temperature under nitrogen for 1 hour. At the end of the reaction dicyclohexylurea (DCU) is removed by filtration.

The solution is washed with 0.1N NaOH and cold water. The organic solution is then dried on anhydrous sodium sulphate and evaporated. After removal of the solvent, the mixture was chromatographed on silica gel eluting with a mixture of dichloromethane/methanol (99.5/0.5).

The compound, after washing first with ether and then with ethanol, has a melting point of 103-106° C.

### EXAMPLE 2

### Synthesis of (Z)-5-Fluoro-2-methyl-1-[[4-(methylsulfide)phenyl]methylene]-1H-indene-3-acetic acid 4-(thioxo-5H-[1,2]dithiol-3-yl)-phenyl ester

Synthesis is performed according to example 1 using sulindac sulfide instead of sulindac sulfoxide.

### EXAMPLE 3

### Synthesis of (Z)-5-Fluoro-2-methyl-1-[[4-(methylsulfone)phenyl]methylene]-1H-indene-3-acetic acid 4-(thioxo-5H-[1,2]dithiol-3-yl)-phenyl ester

A 1N solution of dicyclohexylcarbodiimide (DCC) in chloroform (1.5 ml) is added to 25 ml of a chloroform solution containing 5-(p-hydroxyphenyl)-3H-1,2-dithiol-3-thione (303 mg, 1.34 mmol) prepared as described in example 1, sulindac sulfone (500 mg, 1.34 mmol), and a catalytic amount (7.8 mg) of 4-dimethylaminopyridine (DMAP).

The mixture is stirred at room temperature under nitrogen for 1.30 hour. At the end of the reaction dicyclohexylurea (DCU) is removed by filtration.

The solution is washed with 0.1N NaOH and cold water. The organic solution is then dried on anhydrous sodium sulphate and evaporated. After removal of the solvent, the mixture was chromatographed on silica gel eluting with dichloromethane.

The compound, after washing with ether, has a melting point of 197.3-198.8° C.

### EXAMPLE 4

### Synthesis of (Z)-5-Fluoro-2-methyl-1-[[4-(methylsulfinyl)phenyl]methylene]-1H-indene-3-acetic acid 2-methanesulfonylsulfanylethyl ester

Sodium sulphide nonahydrate (4 g; 16.32 mmol) is suspended in 12 ml of anhydrous ethanol at −15° C. To this suspension is added dropwise a solution of methansulfochloride (1.4 ml, 17.96 mmol) in 3.2 ml of anhydrous ethanol. After complete addition the suspension is kept overnight under stirring. The mixture of reaction is filtered and the solution evaporated to dryness.

Pure sodium methanthiosulfonate is obtained after crystallization of the residue by ethanol. The sodium methanthiosulfonate (690 mg, 5.14 mmol) is suspended in ethanol and 2-bromoethanol (0.76 ml, 10.28 mmol) is then added dropwise to the suspension. After the complete addition the reaction mixture is refluxed for 7 hours.

After cooling, the product is filtered to remove NaBr. The ethanol solution is evaporated, and the residue is dissolved in CHCl₃ and extracted with water.

The aqueous solution is evaporated to dryness, tetrahydrofuran (THF) is added to the residue and the obtained suspension is filtered.

The THF solution is evaporated to obtain a pale yellowish oil (methanethiosulfonic acid S-(2-hydroxyethyl) ester, HEMTS).

HEMTS (400 mg, 2.56 mmol), sulindac sulfone (912 mg, 2.56 mmol) and dimethylaminopyridine (DMAP) (15 mg) are added to a 1N solution of dicyclohexhylcarbodiimide (DCC) in methylene chloride (2.6 ml). The reaction is stirred at room temperature, under nitrogen for 1.5 hours.

The mixture is filtered, and the dichloromethane solution is extracted with 1N HCl, water, saturated solution of sodium bicarbonate and water.

After evaporation of the solvent the residue was chromatographed on a silica gel column, eluting with dichloromethane containing 0.5% of methanol.

The compound has a melting point of 118.5-119.5° C.

### EXAMPLE 5

### Activity

Aberrant crypt foci (ACF) were induced in Sprague-Dawley rats by using 1,2-dimethylhydrazine (DMH). Test agents or vehicle were then administered for 3 weeks, twice daily through orogastric gavage.

At the end of this period, the number and multiplicity of ACF were determined.

The agents tested at equivalent anti-inflammatory doses were: sulindac (standard NSAID), celecoxib (specific COX-2 inhibitor) and sulindac sulfone (no known COX activity) sulindac sulfone derivatives of Examples 3 and 8.

All test agents reduced the number of ACF. There was a reduction of 46% with sulindac, 22% with celecoxib, 36% with sulindac sulfone, 45% with sulindac sulfone derivative of Example 3 and 60% with sulindac sulfone derivative of Example 8.

### EXAMPLE 6

### Activity and Safety

Thirty male rats (150-200 g) were divided into three groups of 10 animals/group and treated with vehicle, sulindac sulfoxide (20 mg/kg) and the compound example 1 (36 mg/kg) for five days once daily by gavage.

At the fifth day survival animals were sacrificed 6 hr after the treatment by an overdose of ether, and autopsied to detect any pathological changes, particularly in the gastrointestinal tract.

Prostaglandin E2 was evaluated in the liver homogenate by standard enzyme immunoassay procedure as described by Warner T D et al. (J.P.E.T. 2004, 310, 642-7).

The results of this experiment is shown in the following Table 2:

TABLE 2

| | | % gastrontestinal |
|---|---|---|
| | % deaths | ulcers |
| Controls | 0 | 0 |
| Sulindac sulfoxide | 70 | 100 |
| Compound example 1 | 0 | 10 |

Prostanoid formation in liver homogenates of animals treated with compound example 1 or sulindac sulfoxide was always markedly reduced (by 90% or more) as compared to the samples from vehicle-treated animals.

On the whole these results indicate that compound of Example 1 is significantly better tolerated than sulindac while maintaining fully the activity (i.e. the ability to inhibit prostaglandin formation).

### EXAMPLE 7

### Synergy Between Compound of Example 4 and Other Agents in Caco 2 Cells

In order to analyze sulindac derivatives in combination therapies for treatment of colon cancer, the compound of example 4 was tested in Caco 2 cell lines comparatively with other known therapeutic agents at different concentrations.

The results of the treatment with 5-FU, celecoxib and docetaxel in Caco 2 cell are depicted in the Table 3 below reported.

TABLE 3

| | IC 50 |
|---|---|
| Agent | (μM) |
| 5-FU | 28 |
| celecoxib | 60 |
| Docetaxel | 0.005 |
| Compound Ex. 4 | 55 |

In another experiment the compound of example 4 at IC50 concentration was incubated with submaximal concentrations of the anti-tumoral standard agents. A marked potentiation of the anti-proliferative activity was observed.

### EXAMPLE 8

### Synthesis of (Z)-5-Fluoro-2-methyl-1-[[4-(methylsulfone)phenyl]methylene]-1H-indene-3-acetic acid 2-methanesulfonylsulfanylethyl ester

Methanethiosulfonic acid S-(2-hydroxyethyl) ester (HEMTS, prepared as described in example 4) (328 mg, 2.1 mmol), sulindac sulfone (782 mg, 2.1 mmol) and dimethylaminopyridine (DMAP) (12.3 mg) are dissolved in 37 ml of chloroform and are added to a solution of dicyclohexhylcarbodiimide (DCC) (476.6 mg, 2.31 mmol) in chloroform (2.3 ml). The reaction is stirred at room temperature, under nitrogen for 2.5 hours. The mixture is filtered, and the chloroformic solution is extracted with 1N HCl, water, saturated solution of sodium bicarbonate and water. After evaporation of the solvent, the residue was chromatographed on a silica gel column eluting with a cyclohexane/ethyl acetate mixture (from 80/20 to 65/35).

The obtained compound, after washing with ethyl ether, has a melting point of 120.4-121.1° C.

### EXAMPLE 9

### Synthesis of (Z)-5-Fluoro-2-methyl-1-[[4-(methylsulfide)phenyl]methylene]-1H-indene-3-acetic acid 2-methanesulfonylsulfanylethyl ester

Synthesis is performed according to example 8 using sulindac sulfide instead of sulindac sulfone.

1. Sulindac derivatives having formula: 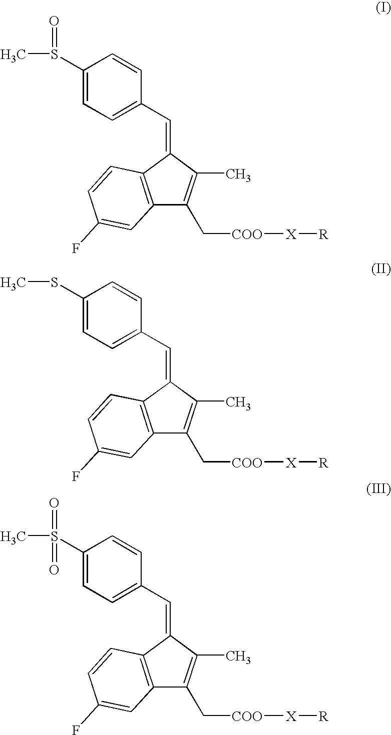
wherein X is absent or, if present, is selected among ethyl-, propyl-, butyl diols, dialkylamine, hydroxyalkylamine or linking groups such as esters, amides, imides, sulfonamides, azo groups, carbamates, carbonates, anhydrides, acetals, thioacetals and R is selected from the group comprising 5-(p-hydroxyphenyl)-3H-1,2-dithiol-3-thione, 1,3-dithiol-2-thione-5-carboxylic acid, 3-thioxo-3H-1,2-dithiole-5-carboxylic acid, 3-thioxo-3H-1,2-dithiole-4-carboxylic acid hydroxyalkylmethanthiosulfonates, N-acetyl-penicillamine, S-allyl-cysteine, bucillamine, carbocysteine, cysteamine, cystathionine, homocysteine, mecysteine, methionine, pantetheine, penicillamine, penicillamine disulfide, thioacetic acid, thiodiglycolic acid, thioglycolic acid, thiolactic acid, 2-thiolhistidine, thiomalic acid, thiosalicylic acid, tiopronin.

2. Sulindac derivative according to claim 1 which is (Z)-5-Fluoro-2-methyl-1-[[4-(methylsulfinyl)phenyl]methylene]-1H-indene-3-acetic acid 4-(thioxo-5H-[1,2]dithiol-3-yl)-phenyl ester.

3. Sulindac derivative according to claim 1 which is (Z)-5-Fluoro-2-methyl-1-[[4-(methylsulfide)phenyl]methylene]-1H-indene-3-acetic acid 4-(thioxo-5H-[1,2]dithiol-3-yl)-phenyl ester.

4. Sulindac derivative according to claim 1 which is (Z)-5-Fluoro-2-methyl-1-[[4-(methylsulfone)phenyl]methylene]-1H-indene-3-acetic acid 4-(thioxo-5H-[1,2]dithiol-3-yl)-phenyl ester.

5. Sulindac derivative according to claim 1 which is (Z)-5-Fluoro-2-methyl-1-[[4-(methylsulfinyl)phenyl]methylene]-1H-indene-3-acetic acid 2-methanesulfonylsulfanylethyl ester.

6. Sulindac derivative according to claim 1 which is (Z)-5-Fluoro-2-methyl-1-[[4-(methylsulfone)phenyl]methylene]-1H-indene-3-acetic acid 2-methanesulfonylsulfanylethyl ester.

7. Sulindac derivative according to claim 1 which is (Z)-5-Fluoro-2-methyl-1-[[4-(methylsulfide)phenyl]methylene]-1H-indene-3-acetic acid 2-methanesulfonylsulfanylethyl ester.

8. Sulindac derivatives according to claim 1 for use as a medicament.

9. Use of the sulindac derivatives according to claim 1 for the manufacture of a medicament for treatment and/or prevention of cancer.

10. Use according to claim 9 wherein the cancer is colon cancer.

11. Pharmaceutical composition comprising one Sulindac derivative according to claim 1 optionally with adjuvants and excipients pharmaceutically acceptable.

12. Synthesis of Sulindac derivatives according to claim 1, said synthesis comprising the reaction of the parent compound Sulindac sulfoxide ((Z)-5-Fluoro-2-methyl-1-[[4-(methylsulfinyl)phenyl]methylene]-1H-indene-3-acetic acid), Sulindac sulfide ((Z)-5-Fluoro-2-methyl-1-[[4-(methylsulfide)phenyl]methylene]-1H-indene-3-acetic acid) or Sulindac sulfone ((Z)-5-Fluoro-2-methyl-1-[[4-(methylsulfone)pheny]methylene]-1H-indene-3-acetic acid, respectively, with a substance (R) containing organic sulfur, active on nuclear transcription factors, the latter or the parent compound eventually modified with a bi-functional linker group (X), said substance (R) and the bi-functional linker group (X) as defined according to claim 1.
